(19) United States
(12) Patent Application Publication
(10) Pub. No.: US 2021/0220594 A1
(43) Pub. Date: Jul. 22, 2021

(54) VIDEO-ENDOSCOPIC INTUBATION STYLET

(71) Applicants: UNIVERSITÄT ZÜRICH, Zürich (CH); ETH ZÜRICH, Zürich (CH)

(72) Inventors: Peter BIRO, Feldmeilen (CH); Quentin BOEHLER, Zürich (CH); Christophe CHAUTEMS, Zürich (CH); Bradley James NELSON, Zumikon (CH)

(21) Appl. No.: 17/262,470

(22) PCT Filed: Jun. 25, 2019
(86) PCT No.: PCT/EP2019/066892

§ 371 Date: Jan. 22, 2021

(30) Foreign Application Priority Data
Jul. 25, 2018 (EP) 18185411.8

(51) Int. Cl. A61M 16/04; A61B 1/005; A61B 1/267

(52) U.S. Cl. CPC A61M 16/0488; A61B 1/0052; A61B 1/267; A61M 2205/502; A61M 2205/332; A61M 2205/505; A61M 2205/3313; A61M 16/0418

(57) ABSTRACT

An intubation stylet includes a shaft with a bendable end section having a tip and a camera at the tip for providing an image of a part of the environment of the tip. The stylet includes a control unit for control of the bending motion of the end section. The control unit is adapted to work under at least two operation modes. In a first of the two operation modes the bending motion of the end section is guided automatically by an orientation of the tip. In a second operation mode, the bending motion of the end section is controlled by manual control of a user without an automatically guidance. The control unit is further adapted to operate in the second operation mode when the first operation mode is inactive.

### BACKGROUND AND SUMMARY OF THE INVENTION

Exemplary embodiments of the invention relate to a video-endoscopic intubation stylet for the guided tracheal intubation with an endotracheal tube, which is automatically guided by light emanating from the trachea or which is manually guided by an image transmitted from a camera at the tip to a video monitor.

Intubation stylets are known in the art. In the current state of the art, numerous guiding methods for conventional and digital intubation stylets have been developed.

Conventional intubation stylets are inserted through the mouth and are guided by the user. A camera is positioned at the tip surface of the end section. The tip can be orientated mechanically by steering the intubation stylet with the help of a handle that is a part of the handpiece. Sometimes the decision in which direction the tip should be orientated is not very easy. Therefore an automatically guided intubation stylet has been developed.

One example of an automatically guided intubation stylet is described by US 2011/0178369 A1. The intubation stylet has a guide control device for an automatic orientation of the tip of the intubation stylet towards a signal generated by a trachea identifier source.

Until the tip of the stylet passes the vocal cords, the stylet is steered in automatic mode (by directing the tip towards the light). After passing the vocal cords the user switches the stylet to manual mode and steers the tip further into the subglottic trachea by viewing the image transmitted from the camera at the stylet tip to an external video screen.

Other documents describing possible variations of an intubation stylet are described in WO 2014184796 A2; CN 201256953 Y; US 2015096556 A1 and US 20100087711.

Exemplary embodiments are directed to an intubation stylet with an automatically infrared light guided bendable end section. The detectable wave length of the light is chosen that way that malfunctions due to distracting signals are prevented.

An inventive intubation stylet comprises a shaft having a bendable end section with a camera in the tip. The camera is adapted to provide an image of a part of the environment of the tip. This image could be, for example, a picture or most preferably a video image. The image might be visualized by the user or automatically interpreted by an image recognition software that recognizes typical features of the upper airway such as the pharynx, hypopharynx, larynx, glottis, vocal cords, trachea, and bifurcation.

The intubation stylet is preferably used for the guided tracheal intubation with an endotracheal tube, which is railroaded on its shaft.

The tip may preferably comprises a sensor element for detection of the direction of an incoming light signal for example from an external source such as a light source, preferably a near infrared (NIR)-light source.

The sensor element can be analogue to a “primitive eye”, comprising an array of photo diodes positioned at a distal end of the end section on the frontal surface of the tip. The photo diodes are suitable to detect differences of light intensity, so that the signals emitted by a light source can be classified into signals of photo diodes positioned in a concave frontal surface of the stylet tip that is surrounded by a wall that produces a shadow. A shadow will occur when the frontal surface is not positioned along the vector of a light signal, which means that the signal does not fall perpendicular on the frontal face of the stylet tip. Alternatively or additionally to the aforementioned feature, a video camera is positioned at the frontal surface of the tip of the intubation stylet, from which the image is interpreted by the processor that way, that the direction from where the guiding light comes is recognized. This interpretation of the image can be automatic by an image recognition software or by viewing the image on a video screen by the user.

In one embodiment the bending motion of the end section is guided automatically towards a direction by an orientation of the tip with the aforementioned sensor element at the tip towards an incoming signal, such as a light signal, preferably an infrared-light. By detecting this signal the sensor element and the control unit may generate a control signal to an actuated pull system which will guide the tip towards the incoming signal.

In a second embodiment the bending motion of the end section is guided automatically towards a direction by an image recognition, preferably a video recognition, by the camera. In this case a further sensor at the tip of the stylet is not needed.

The intubation stylet is further provided with a control unit for controlling the bending motion of the end section. A bending motion according to the current invention also comprises a tilting and/or rocking motion of segments of the end section relative to each other.

The insertion of the stylet inside the trachea is preferably performed manually by the user in order to avoid damages, therefore the bending motion is only controlled by the two modes but not the lateral movement or in other words the advancement of the stylet inside the trachea, which is always performed manually.

The control unit is adapted to work under at least two operation modes. The control unit can be also fitted with a monitor screen. The control unit and the screen can be adapted to the shaft of the intubation stylet. It is, however, also possible that each of these parts or both parts can be separate elements and not directly connected to the shaft. The control unit and/or the screen can interact with the rest of the intubation stylet via a wire or by wireless communication.

The control unit can preferably be equipped with a microprocessor and with at least one database. The database manages a set of stored data, such as parameters or images for the comparison of light intensities and for a recognition of the advance of the intubation stylet during its insertion.

The microprocessor can compare measured values with the stored data and adapt the orientation of the tip automatically to receive a maximization of a light intensity to be detected.

This automatic orientation of the tip by a bending towards a maximum of light will be performed in a bending motion of the end section of the stylet.

While the orientation of the tip will usually be done automatically in the first operation mode, the advance of the intubation stylet inside the trachea can be done either manually or automatically, whichever type advancement is preferred.

The automatic orientation in the first operation mode enables a quicker identification of a position so that the intubation stylet can be easier advanced further.

In a second operation mode the bending motion of the end section is controlled by manual control of a user. Manual control can be exerted directly by moving a joystick at the proximal end of the intubation stylet or a generation of a control signal by the user with the help of the video data of the tip, wherein the control signal can be transferred, e.g., by wireless data transfer to a unit, such as an electric motor (actuator) inside the shaft. The motion of the tip can be guided automatically or by the user according the video signal from the camera at the tip.

The control unit is further adapted to operate either in the first operation mode or the second operation mode. So the first operation mode where the orientation is guided automatically by a signal from an external source is inactive when the second operation mode is performed.

The first operation mode could be according to a first aspect of the invention a fully automatic mode or according to a second aspect of the invention a user-corrected-automatic mode. The user-corrected-automatic mode is a combination of automatic mode with the guiding of the end section by at least one sensor but where a manual correction of this automatic guidance by the user is possible, wherein in the fully automatic mode the orientation of the end section is guided only by at least one sensor without the possibility of a correction of the user, for example by an overriding action.

Therefore the control unit is further adapted to operate the first mode wherein the bending motion of the end section is guided automatically by an orientation of the tip towards the direction of the signal on the basis of at least one signal generated by the sensor element but can be corrected manually by the user. This signal should be understood as a control-signal, which is generated after the detection of the incoming signal, such as a light signal or in combination with the detection of other signals, such as resistance signals.

In general, the stylet is always provided with a first operation mode, being an automatic mode or a user-corrected automatic mode, and with the second operation mode being a manual mode.

The user can decide when the manual mode is needed. There could be the option that the user decided not to switch from the user-corrected automatic mode into the manual mode. However, it is recommended for the most cases that the manual mode is activated at a certain moment or position of the tip during the introduction of the stylet.

In both embodiments the second operating mode, the manual mode, is without any automatic orientation of the end section.

The inventive intubation stylet has better control and less risk of damage or injury compared to the state of the art.

It is advantageous when the switch from the first mode to the second mode is performed automatically, for example when a certain position in the body is reached. This can, for example, be achieved by a picture comparison of the video image.

The control unit can be adapted for an overriding of the first mode by a manual control of the user. Therefore, at all times, the user has the control of the stylet and its movement. In the context of the current invention an overriding could be a deactivation of the automatic orientation of the end section or a user-corrected automatic mode as previously described

The overriding can preferably be triggered by a user decision and/or by a collision of the end section with an obstacle, preferably by sensing a resistance by a force sensor. Triggered in the context of the current invention means that the overriding can be started either by the user or by the control of the mechanical resistance.

It is of advantage when the bendable end section of the shaft comprises a plurality of joint segments. One of these segments is the tip. Each of these segments are connected to a neighboring segment by a joint. The most proximal segment opposite to the tip can also be connected to a more rigid part of the intubation stylet shaft by a joint. The rigid part of the shaft can also be bendable but preferably consists of only one elongated piece.

Each segment can be short compared to the rigid part of the shaft, preferably at least less than 10% of the length of the rigid part of the shaft. Each segment can have a tubular form. Each segment preferably has a longitudinal axis. Each segment has tiltable joints to neighboring segments. The joint between two segments can be adapted in a way such that the longitudinal axis of the segment and the neighboring segment define a bending angle. The expressions for bending angle, tilting angle and rocking angle are synonyms according to the current invention.

The bending angle between two neighboring segments is preferably increasing towards the tip of the intubation stylet. This way the joints have increasing mobility towards the tip of the bendable end section. The non-constant curvature reduces the risk of an undesired orientation of the tip and of an undesired bending movement of the end section.

Further, the end section of the shaft can better adapt to the anatomical shape of the upper airway including pharynx, hypopharynx, larynx and trachea.

It is advantageous that the bending motion of the end section can be performed in all circular directions by a control of at least 3 tension cables that extend at least over a part of the length of the end section of the shaft. These tension cables can be connected directly or via actuators to a joystick or another suitable handheld device. Due to mechanical solution for the control of the bending motion, the user can feel a resistance when the motion of the tip is hindered, so that the risk of injuries is reduced. Alternatively or additionally to the aforementioned feature, due to electronically created and transmitted of a haptic feeling for the joystick handling hand the user can feel a resistance when the motion of the tip is hindered, so that the risk of injuries is reduced.

It is advantageous when the tip of the intubation stylet comprises a front surface and a housing, preferably a tubular housing. The camera is either at the very end of the tip and extending radially from the housing, or it is in a back layered position to the front surface or it is extending radially from a housing of one of the other segments, especially the neighboring segment of the tip, of the end section. The back layered position of the camera can visualize the entry of the stylet tip into the larynx.

The intubation stylet can preferably be provided with a screen, which is preferably connected to the shaft or can alternatively be positioned separately and connected by a wire or by wireless transmission. The screen is an output-device for an image, preferably a picture or a video, provided by the camera. The screen can provide a video or picture in both operating modes. In the second operating mode the screen helps the user for a better control of the orientation of the tip.

In a preferred embodiment of the current invention, the sensor element can work like a primitive eye. In this configuration the sensor element comprises a number of photo diodes positioned at a distal end of the tip under a transparent cover for the protection of the photo diodes, wherein the transparent cover is framed by a wall section of the housing, which will provide a shadow when a light signal comes from an inclined direction. The control unit will then try to find the orientation of the tip where the most amount of light is detected by the photo diodes. Alternatively or additionally to the aforementioned feature, a video camera is mounted onto the tip of the endoscope instead of or additionally to the photo diodes.

The transparent cover provides the front surface of the tip wherein the front surface has a concave form. The radius of the concavity may be in the range 1 to 6 outer diameters of the endoscope. This is advantageous to increase the area in which the shadow is formed. Therefore, even small deviations from the optimal orientation can be detected. Alternatively or additionally to the aforementioned feature a video camera is mounted onto the tip of the endoscope instead of or additionally to the photo diodes.

The tip can further comprise a force sensor for the detection of a contact. Due to the pressure sensor and the camera image, the user can differentiate between different materials that are probable in contact with the tip, such as tissue or harder material such as cartilage of the upper airway.

The shaft, especially its end section, can preferably be provided with at least one longitudinal elastic rod that extends at least partially through the end section with parallel orientation to the axis of the end section. The elastic rod enables a retractable movement of the tip and the entire shaft, for example when the force sensor detects an obstacle.

The shaft, either the end section or the rigid part, can be provided with a recognition sensor, preferably a photo diode, which is positioned at or in an anterior surface of the shaft at a predefined distance of the front surface for the recognition of the depth of the intubation procedure. Alternatively or additionally to the aforementioned feature, an image recognition software could define the tip position by recognizing characteristic anatomical features inside of the upper airway.

An intubation system according to the invention comprises the previously described inventive intubation stylet in the embodiment with a sensor at the tip of the stylet as a first module of the intubation system which can be at least partly inserted through the mouth of a patient, and a signal source, especially a light source, as a second module of the intubation system which can be attached externally in midline at the suprasternal neck region of a patient, for example by a necklace, adhesive stripes, a choker or other means. The light from the light source has a wavelength in the NIR spectrum between 750 and 1400 nm.

In a preferred embodiment, the signal source emits the signal that corresponds to the aforementioned incoming signal, wherein the signal is a NIR-signal (near infrared signal). The wavelength of the signal can preferably be in the infrared spectrum (750 nm to 1400 nm). The intensity of the signal emitted by the signal source can be varied by the user or by the control unit of the light source device.

A method for the performance of the inventive intubation system, comprises a sequence of at least two operation modes as method steps:
a. in a first of the two operation modes the bending motion of the end section is controlled by an autonomous orientation of the end section towards the direction of the light signal of a on the basis of at least one signal generated by the sensor element; and wherein
b. in a second of the two operation modes the bending motion of the end is controlled by manual control of a user.

The control unit is further adapted to operate in the second operation mode when the first operation mode is inactive.

The intubation stylet is switched from the first operation mode into the second operation mode at a predefined distance when the tip is approaching the signal source. After passing that specific insertion depth, the first operation mode of the intubation stylet can preferably be blocked to prevent injuries.

The switching between the operation modes can optionally or alternatively be manually triggered by the user.

According to a preferred embodiment of the invention the bending motion of the end section, such as each bending angle of two neighboring segments of the end section, can be measured, preferably continuously measured, and displayed at the screen. For this reason each segment or link can be equipped with a suitable sensor element to determine the local bending angle.

An advantageous embodiment of the inventive method comprises at least the following steps:
a) an image of a part of the environment of the tip is recorded by the camera at an actual position;
b) the image is compared with a database representing a predetermined image at a predefined position by the control unit
c) determination of a value representing the actual position and/or orientation of the tip based on the comparison of step b); and
d) switching from the first in the second operation mode if the determined value of step c) has reached, exceeded or under-cut a threshold value.

Alternatively or additionally to step b) the anatomical features, especially inside the trachea, are localized using computer vision by the control unit.

A further inventive method for the performance of the inventive intubation stylet, comprises a sequence of at least two operation modes as method steps:
a. in a first of the two operation modes the bending motion of the end section is controlled by an autonomous orientation of the end section; and wherein
b. in a second of the two operation modes the bending motion of the end is controlled by manual control of a user.

The control unit is further adapted to operate in the second operation mode when the first operation mode is inactive.

The intubation stylet is switched from the first operation mode into the second operation mode by an image recognition method with the following steps:
a) an image of a part of the environment of the tip is recorded by the camera at an actual position;
b) anatomical features, especially inside the trachea are localized using computer vision by the control unit;
c) determination of a value representing the actual position and/or orientation of the tip based on the comparison of step b); and
d) switching from the first in the second operation mode if the determined value of step c) has reached, exceeded or under-cut a threshold value.

The aforementioned method, especially the localization, can be performed by object classifiers such as a Haar-Cascade Classifier working with the Viola Jones method. An object classifier is adapted for the gathering of visual data, such as a high number of images of the trachea or the glottis. The objects of the recorded images are classified. The cascade, as a set of convolution kernels, is chosen by their best fit with the objects. These kernels can be detected and chosen by a machine learning program, which is trained based on the collected visual data.

An individual kernel cascade classifier is created after this learning step for the use in the inventive stylet. This individual classifier can be used for different anatomical features. The computer convolves the chosen kernels with an image detected by the camera of the stylet, and it gives a statistical probability of if it found the object of interest, such as the glottis, and where the object is in the image.

The described method is, of course, just one example for a localization. Other methods like histograms of oriented gradients, convolution neural networks and You Only Look Once, also called “YOLO” as a real time object detection are all different computer vision techniques that we could possibly be used for within the scope of the method according to the invention.

The localization of anatomical features, especially inside the trachea, by using computer vision by the control unit can be further described by a localization of anatomical features using computer vision based object detection, especially real-time object detection.

### SUMMARY OF THE DRAWING FIGURES

An advantageous embodiment of an inventive intubation system is further explained in detail by a drawing. Specific parts of the embodiment can be understood as separate features that can also be realized in other embodiments of the invention. The combination of features described by the embodiment shall not be understood as a limitation for the invention:

FIG. 1: a schematic view of a first example of an inventive intubation stylet;

FIG. 2 a schematic perspective view of a part of the intubation stylet of FIG. 1;

FIG. 2a a schematic perspective view of the camera in FIG. 2;

FIG. 3 a cross-sectional view of an end section of the intubation stylet of FIGS. 1 and 2;

FIG. 4 a cross-sectional view of the retina at the tip of the intubation stylet of FIG. 1-3;

FIG. 5 a cross-sectional profile view of the tip of the intubation stylet of FIG. 1-4;

FIG. 6 a schematic view of the bendable end section of the inventive intubation stylet of FIG. 1;

FIG. 7 a perspective view on a signal source as a part of the inventive intubation system;

FIG. 8 a schematic view of a control unit 3 for the intubation stylet of FIG. 1;

FIG. 9 a screen-view of the intubation stylet of FIG. 1;

FIG. 10 a schematic view of a second example of an inventive intubation stylet;

FIG. 11 a schematic view of the bendable end section of the inventive intubation stylet of FIG. 10;

FIG. 12 a cross-sectional view of the end-surface of the tip of the intubation stylet of FIGS. 10 and 11;

FIG. 13 a cross-sectional view of a section, especially a neighboring section, of the intubation stylet of FIG. 10-12; and

FIG. 14 a screen view in an example of procedure. Step 1: manual mode, Step 2: autonomous mode without user correction, Step 3: autonomous mode with user correction.

### DETAILED DESCRIPTION

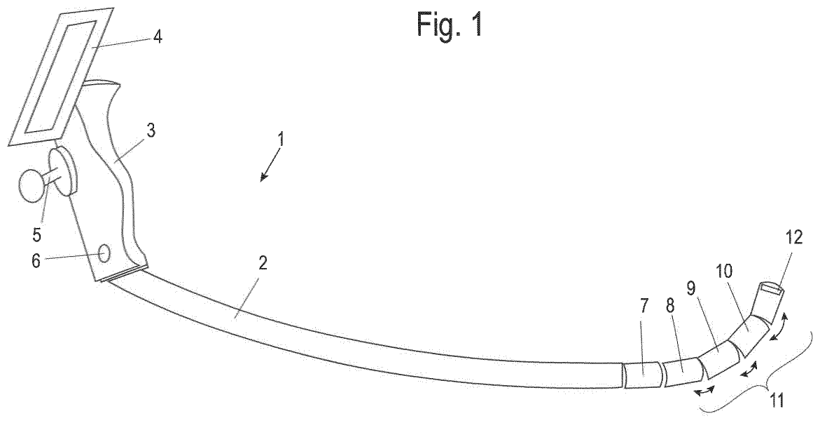

FIG. 1 illustrates a first embodiment of an inventive intubation stylet 1 according to the invention. The intubation stylet 1 comprises a shaft 2, a control unit 3 with a screen 4 for visualization of the position of a tip 12 of the shaft 2 of the intubation stylet 1.

The tip 12 is part of a bendable end section 11 comprising a plurality of linked segments 7-10 and 12. Each segment can be bend to a certain angle to a neighboring segment.

Each segment defines a longitudinal axis. A bending angle is defined between two neighboring segments or between the rigid part of the shaft 2 and the first neighboring segment 7 after the rigid part of the shaft. The bending angles between two neighboring segments 7-8, 8-9, 9-10, 10-12, increases from rigid part of the shaft 2 to the tip 12.

The control unit 3 includes a control button for switching between to operation modes of the intubation stylet 1. The control unit 3 further includes an actuating element 5, such as a joystick, for the bending movement of the tip 12 or any other orientation of the tip 12 relative to the shaft 2.

The diameter of end section 11 comprising the linked segments 7-10, 12 is 80-120% of the diameter of the shaft. The diameter of the shaft can preferably have less than 1 cm, preferably 3-7 mm.

The definition of the rigid part of the shaft according to the invention should not be understood in a way that the rigid part could also be preferably slightly flexible or semi-rigid.

The end section 11 can preferably have a length of less than 30% of the length of the rigid part of the shaft, most preferably between 10-25% of the length of the rigid part of the shaft 2.

The shaft 2 or the control unit 3 can comprise an electrical drive or an actuated pull system, such as a mechanical pull system, for the control of the orientation of the tip 12 by the control unit 3.

The stylet can most preferably be a cable driven endoscope with the cables being connected to actuators. The control signals of the actuators are either computed to steer the stylet toward the detected trachea in the automatic mode (first mode), or use the input from the joystick in the manual mode (second mode). Therefore, a system of cables connected to actuator is defined as an actuated pull system in the context of the current invention.

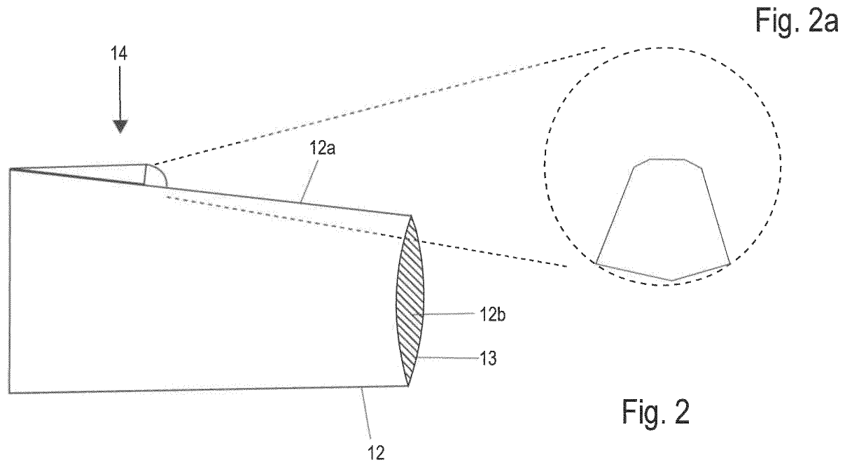

An embodiment of a tip 12 is shown in FIG. 2-5.

FIG. 2 shows a tip 12 comprising a housing 12a with conical shape. A retinal lens 13 is provided at the front surface 12b of the tip 12. The tip 12 is provided with a first camera 14 positioned at and extending radially from the conical wall surface of the housing 12a.

Due to the slope of the conical shaped housing 12a with a decreasing diameter towards the front face 12b of the tip 12′, the view angle of the camera is broader compared to a housing of a tip with cylindrical shape. However, it could be understood that a housing could only have a sloped surface extending from the camera 14 to the front surface 12b of the tip could have the same effect as a conical surface.

The view of the camera 14 comprising a partial view of the wall surface is schematically shown in FIG. 2a. The camera 14 can be a CCD camera (charged coupled device). The camera can be used as a light detector for a guided orientation of the tip towards a light source during the axial movement of the intubation stylet or for providing a digital image.

The segments 7-10 and 11 can be connected towards each other by a joint, preferably an articulated joint allowing a movement of the segments in all directions within a limited range, such as a ball joint.

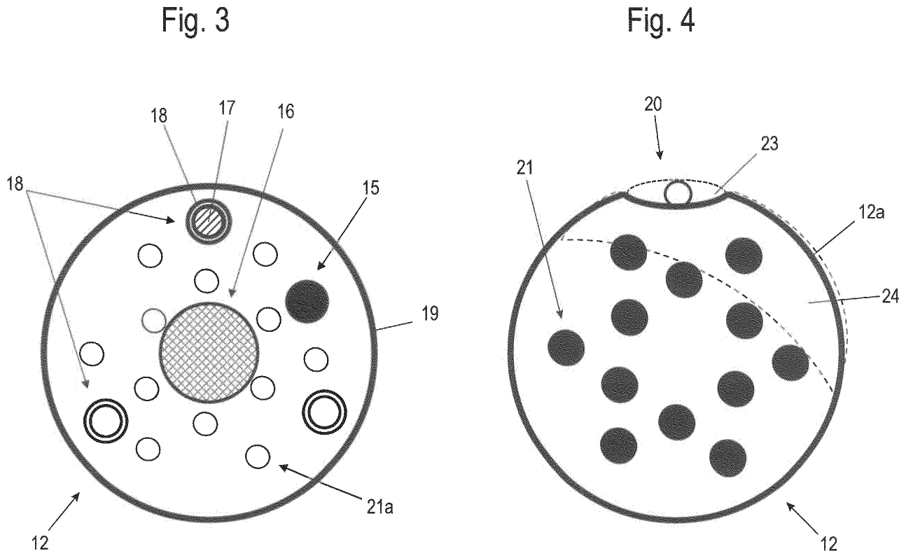

The shaft 2, the segments 7-10 but also the tip 12 can be provided with channels 18 that extend parallel to a longitudinal axis of the housing 19 of the shaft 2 but also of housings of the segments 7-10. As shown in FIG. 3 the rigid part of the shaft 2 can provided with three of these channels 18. Tension cables 17 are positioned in the channels 18. Three cables is the minimum number of cables to achieve the bending in two directions, but more than three cables can be used in practice. Therefore, more than three cables/channels can be considered.

The cables 17 are part of a pull system and can be directly connected to the control unit or to the electrical drive or any other suitable actuator. Electromagnets can also interact with the tension cables. The channels 18 with the tension cables 17 are spaced apart from each other and from the longitudinal axis of the shaft 2 in a symmetrical manner. It could be easily understood that the tension cables 17 can preferably also extend in the same way through the other segments 7-10 and partially also in the rigid part of the shaft 2 and in the tip 12.

Depending on the tension at the cables 17, the segments 7-10 and 12 (i.e., including the tip 12) will bend in a direction, wherein the direction and the bending angle can be controlled by the actuating element 5, for instance a joystick.

At the center of the cross section the shaft 2 comprises an elastic rod 16 extending longitudinal through the tip 12 and preferably also through the other segments 7-10 of the bendable end section 11.

In a preferred embodiment, the segments 7-10 can be at least partly slidable into each other or inside the rigid part of the shaft, for example in a way that all segments have a conical housing.

The elastic rod 16 is retractable. It can be a part of a retraction or recoiling system so that the length of the end section 11 is variable, preferably in a telescopic manner. Due to the retractable elastic rod 16, it is possible to retreat the tip 12 from an undesired position.

A signal transmission cable 17 from the first camera is shown in FIG. 3. As shown in FIG. 4, the retina 13 comprises a plurality of photo-diodes 21 for the generation of an image of the environment in front of the front surface 12b of the tip 12.

Therefore in FIG. 3 a plurality of signal transmission wires 21a are shown, that extend from the photo-diodes 21 to the control unit 3 with the screen 4.

FIG. 4 illustrates the terminal segment of first camera 14. In FIG. 4 housing 12a is provided with an arcuated surface section 23 that extends from the camera 14 to the front surface 12b. This arcuated surface section provides a better view angle for the camera 14. A further light detection can be provided by the photo diodes of the retina 13. A light signal 22 from a non-depicted light source will provide a shadow 24 on the surface of the retina 13. As shown in FIG. 4, some photo-diodes 21 will be located inside the shadow and will therefore measure another light intensity than the photo-diodes 21 outside of the shadow. Due to the fact that the angle and the area covered by the shadow depends on the direction of light, the retina 13 is able to determine the direction of the incoming light. The control unit 3 can calculate a movement in orientation towards the light and can control the tension of the tension cables 17 so that the tip 12 will bend, for example, in a way that the shadow area is minimized.

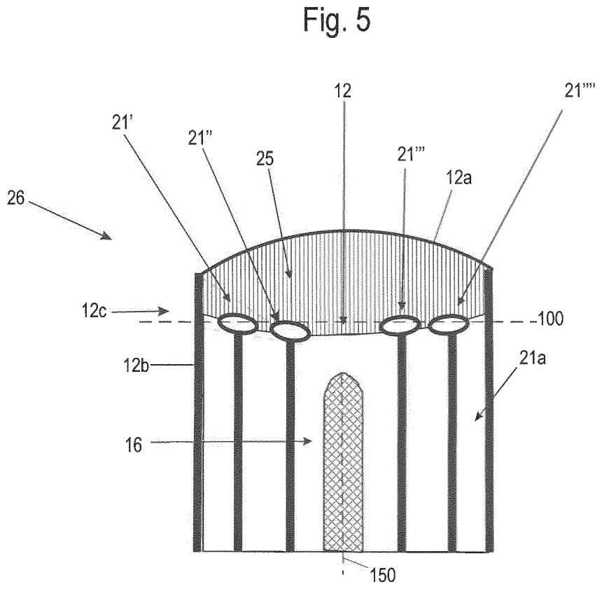

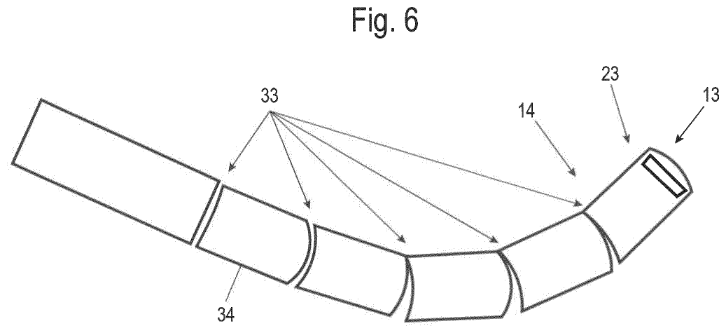

The principle can be explained in FIG. 5. The photo-diodes 21 defining at least one plane 100 perpendicular to the longitudinal axis of the tip 12. The photo diodes are covered by a transparent cover 25. The housing 12a of the tip 12 defines a frame 12c for the transparent cover 25, which extends in a direction parallel to the longitudinal axis 150 of the tip 12. The frame 12c will produce the shadow, when a light source 26 does not shine perpendicular to the front face 12b of the tip 12.

As shown in FIG. 5 the photo diode 21′, which is positioned ipsi-lateral to the shadow 24, will detect the lowest light intensity and the photo diode 21′″,′ which is positioned contra-lateral to the shadow, will detect the highest light intensity. It might be clear from FIG. 5 that the photo diodes being positioned closest to the frame section 12c of the housing 12a will detect smaller incidence angles of the light source.

The housing 12a can comprise a cylindrical wall and an inner polymer material such as potting material or a cast material.

In FIG. 3 or FIG. 5 the signal transmission wires 21a from the retinal photo diodes 21 a signal transmission wire 15 for the optional second camera and the elastic rod 16 are also shown.

In the preferred embodiment of FIG. 5 the transparent cover 25 has a front surface, which is also the front surface 12b of the tip 12. The front surface of the transparent cover 25 is concave. This is of advantage to increase the direction dependency of the light intensity difference detected by the photo diodes 21.

The bendable end section 11 of the shaft 2 is shown in FIG. 6. Joints 33 between the segments 7-10 have increased mobility towards the tip 12. In an alternative embodiment of the invention the segments can have a non-constant curvature by gradually lessening of the resistance to the bending towards the tip 12.

A combination of the manual control and the increased possibility of bending gives the user the opportunity to look around in a more extend way in case that he would need a bit more orientation or in the case that he has discovered another obstacle such as cancer, ulcer, boil and the like, which might be of interest for a further observation.

The camera 14 is slightly protruding from the housing 12a of the tip 12. The camera 14 will preferably send the image of the environment in front of the tip 12, while the retina 13 is provided for the detection of the direction of light and in combination with the control unit 3 to provide a controlled phototropism of the end section 11 of the shaft 2.

Alternatively or additionally to the camera 14, the retina 13 can also be provided with a second camera producing an image of the environment of the tip. The second camera can provide additional information to the image produced by the first camera 14.

An inventive intubation system further comprises a signal source, such as a light source, for emitting the incoming signal for the orientation of the tip in one of the aforementioned embodiments. The light source can preferably provide light with wavelength over 500 nm, more preferably over 600 nm, most preferably infrared light. This light source 30 can have an emission unit 31, comprising for example light producing element, such as an IR-LED, with an emission window, preferably for the contact with the skin, especially for the adaptation near the throat, a control unit. The light source 30 can further comprise a fastening element 32, such as a necklace or a choker.

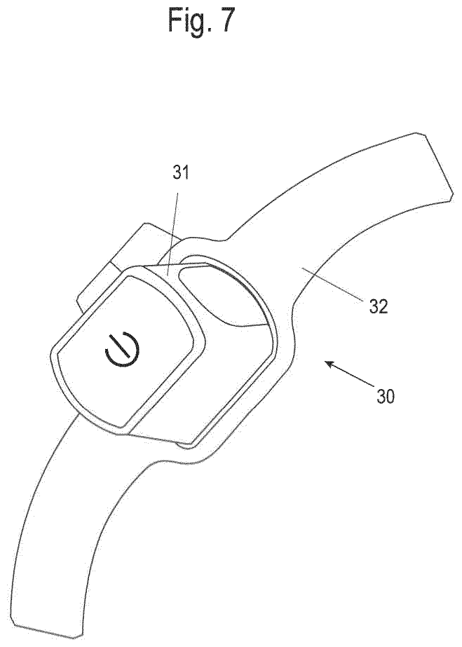

In the intubation system of FIG. 1-7 bending of the intubation stylet is guided by a light signal. It is, however, also possible according to the invention assist the guiding of the intubation stylet by other signals, such as sonic signals or thermal signals provided by a suitable emission unit. With a sonic signal for example a piezo-element can be used in an emission unit and in a signal receiving unit in the tip 12. Light, especially with longer wavelength, however has a better performance due to less perturbations from bones and organic material and is therefore the preferred technique.

The control unit 3 is provided with at least two operating modes.

The intubation stylet is able to operate in these two different self-excluding operating modes:

One first mode is an Autonomous mode AM and a second mode is a Manual mode MM.

The control unit 3 can be provided with a data storage device and a processor. On the data storage device a data set comprising one or more parameters is provided. These parameters can be a light intensity or an image geometry, such as an image of the trachea.

If the camera by comparison of the light intensity detected by the tip or a specific image geometry provided by the camera with the parameters, the camera will switch from the autonomous mode into the manual mode.

In the manual mode the bending of the tip 12 is controlled by the user and in the autonomous mode the bending of the tip is controlled by the signal provided by the emission unit, such as the light emission unit 31, that is a separate unit to the intubation stylet.

Therefore in the autonomous mode the intubation stylet automatically steers the tip 12 toward the light source 30.

In manual mode, the user can uses a 2-DOF, a two degree of freedom, mechanical interface, e.g., the joystick, to generate a bending angle or angular velocity command of the tip starting from the last configuration of the end section 11 reached in the previous operating mode.

The manual mode can be used as a default mode and autonomous mode can be actively triggered by the user. The joystick can be mounted on a monostable switch so that the mode switching and the manual control can be operated with a single hand.

To this extent, the autonomous mode can be activated by maintaining a monostable system (e.g., a monostable switch) within its unstable state (e.g., inward pressure on the monostable switch). The MM is activated by default when the monostable system is within its stable state (e.g. no pressure on the monostable switch).

An example of procedure can be described:
Generate inward pressure on the switch with a thumb to maintain the autonomous mode.
Release the pressure on the switch and use the joystick to control the configuration in manual mode.

Another solution can be considered for the 2-DOF mechanical interface, e.g., a separate switch close to the joystick to switch between autonomous mode and manual mode.

A further aspect of the invention that can be combined with the further features mentioned above is an at least 2-directional bending motion of the end section 11 of the shaft 2 comprising the tip 12, which will explained more in detail.

The end section 11 can be bent along at least two orthogonal directions. This motion is induced by forces that can be generated by several means that can also be combined.

In first option for a force generation in tip, the forces are directly or indirectly generated in the tip 12 of the end section 11 such as a magnetic, electric and/or thermal actuation.

In a second option for the force transmission to the tip 12, the forces are generated from the proximal end of the shaft with the control unit 3 and transmitted to the tip 12 by mechanical means such as cable actuation or fluidic actuation.

When considering the force transmission to the tip, the 2-directional bending is obtained by transmitting three axial forces along the end section 11 acting in a distal cross-sectional plane according to a regular triangle in 3×60° positions. These three axial forces can preferably be obtained either by three pressurized fluidic chambers or at least three cables guided along hollow channels.

Two modalities are preferably be considered and combined for the bending motion:

An active control for a bending motion will be provided by a control of the forces transmitted or generated at the tip 12 by the means previously described.

A passive bending motion can be generated by an elastic restoring force. This force is provided in the described preferred embodiment by an elastic element within the shaft 2, especially within the end section 11, e.g., by the flexible elastic rod 16. In this case, this elastic force tends to move the end section 11 back to its resting baseline configuration.

The diameter of the rod 16 can preferably decrease towards its end section 11, thus being conical. This way the recoiling force is diminishing towards the tip 16 of the shaft 2.

The aforementioned features can be combined in a preferred manner.

A further aspect of the invention is that bending of the end section 11 of the shaft 2 has a non-constant curvature bending.

This is of advantage for a more suitable adaptation of the end section 11 to the anatomical shape of the hypopharynx. The bending angle between two segments of the end section increases toward its tip. This can preferably be provided by a non-constant curvature.

Considering the steering forces acting on the end section 11, there are different possibilities how the non-constant (spiraloid) curvature bending can be achieved:

A first option can be provided by an association of multiple articulated rigid segments of variable lengths and/or coupling stiffness.

A second option can be provided by an association of multiple articulated rigid segments with joints in-between having increasing maximal moving freedom, e.g., first joint blocks at a certain angle, the following joint at a larger angle and so forth until the most distal joint blocks at the largest angle.

The succession of maximal angles can be defined. A total bending of 90° between the shaft and the last segment may be achieved by the succession of four consecutive maximal joint angles: 12°+19°+26°+33° or other sequences.

The segments of the end section can be conical hollow bodies with increasing angular aperture of the conical inner wall or with a decreasing angular aperture of the conical outer wall of the sequence of the segments of the end sections towards the tip 12.

A third option can be achieved by the use of a continuous flexible tube with decreasing bending stiffness along its axis toward the tip. Such a possibility can be provided by a decreasing concentration of fibers inside a rubber material.

A fourth option can be provided by the diminishing retraction capacity of the conical rod 16.

Such variable stiffness flexible end section 11 can be obtained by a gradient of constitutive material stiffness, and/or by a gradient of cross-sectional area moment of inertia along the axis of the end section 11. This also provides the passive bending motion to the resting position when no forces are generated on the tip 12 of the shaft 2, including the decreasing restoring force toward the tip.

The bending curvature is non-constant and increases along the end section 11 from the proximal to the distal end of end section 11, which can also be called stylet.

This feature advantageously provides a non-constant curvature instead of a circular one, meaning that more pronounced distal bending can be achieved for the same radial shift of the distal tip 12 about its long axis.

A preferred light source 30 is a NIR light source (near infra-red light source) with wavelengths between 750 and 1400 nm.

A further aspect of the inventive intubation stylet is the detection of the direction from where the NIR light source shines. Such detection can preferably be achieved by two ways

A first possibility is the detection of the light source centroid within a single NIR sensitive camera image positioned along the axis of the tip 12 at its distal end.

Additionally or alternatively to the first possibility a “primitive eye” system composed of a grid of NIR sensitive photo-diodes positioned on the distal end cross-section, the front section 12b, of the tip can be used in a second possibility. The retina 13 receive different amounts of light depending on the source location, a marginal wall or frame is provided which produces a shadow in the opposite direction of the light source, and a concave shaped transparent cover at the distal end cross-section of the end section is provided that aims at differentiating the light intensity onto the photo-diodes grid caused by their different orientation.

In the first case, an image-based control is to be implemented preferably in the autonomous mode. It consists in controlling the configuration of the end section so that the NIR light source centroid is centered in the camera image, meaning that the tip has the tendency to orient toward the light source.

In the second case, the location of the light source is to be determined by the different light intensities on each photo-diode. These intensities are related to the shadow projected on the retina and/or on the angle in which the light hits the photo-diodes. By computing the pattern of sensed light intensities by the photo-diodes, the microprocessor calculates the direction from where the light comes. The control strategy in autonomous mode comprises the limitation of the shadow, i.e., having equal intensity on each photodiode, resulting that the tip automatically becomes oriented toward the light source.

The wavelength of the light emitted by the light source can be very specific. Wavelength in the visible light spectrum can preferably be filtered out.

In a further embodiment an optional internal illumination by a light source can be switched off during the short time of the measure so that only the NIR light is present in the spectrum at that moment.

In a further embodiment visible light can be provided by a light source but a specific modulation of the NIR light intensity can be used to detect it in an image.

For the primitive eye, a calibration of the system may preferably be required to map the light source location to the projected shadows, and to the corresponding signals on the photo-diodes.

A brief explanation of the “primitive eye” analogy from a biological is here provided: “a cup” shape of the pit eyes can allow a limited directional differentiation by changing which cells the lights would hit depending upon the light's angle. Pit eyes, which had arisen by the Cambrian period, were seen in ancient snails, and are found in some snails and other invertebrates living today, such as planaria. Planaria can slightly differentiate the direction and intensity of light because of their cup-shaped, heavily pigmented retina cells, which shield the light-sensitive cells from exposure in all directions except for the single opening for the light. However, this proto-eye is still much more useful for detecting the absence or presence of light than its direction; this gradually changes as the eye's pit deepens and the number of photoreceptive cells grows, allowing for increasingly precise visual information.” A further explanation can be found in the Wikipedia-Article “Evolution of the eye”.

The cover of the retina 13 can preferably be a glass cover. The curvature of the preferred concave form of the front surface of the glass cover depends on the distribution of the photo diodes on the tip.

A further aspect of the current invention is the back-layered position of the camera 14.

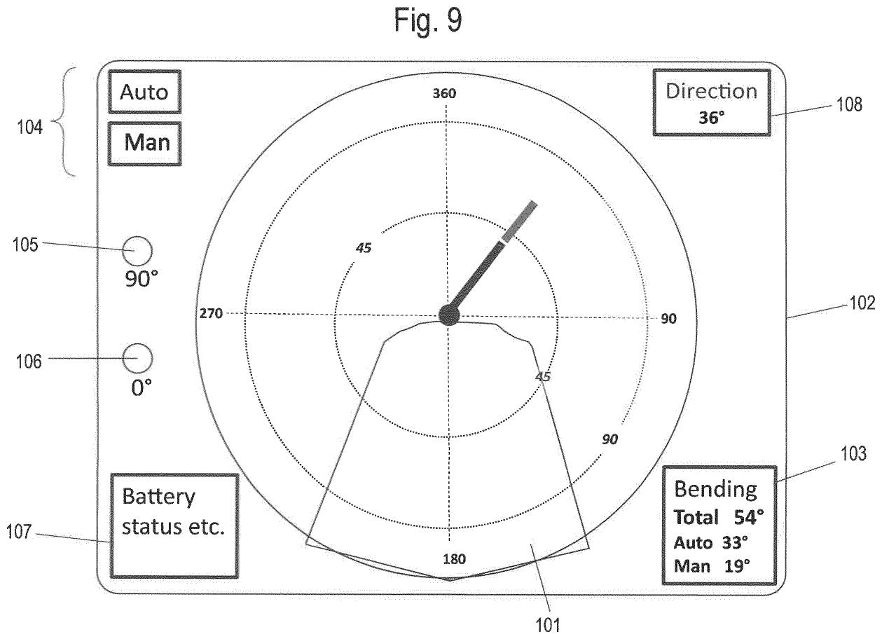

FIG. 9 shows a screen-view 102 of the intubation stylet recorded by the camera 14. A part 101 of the view is covered by the form of the housing 12a. For the user this part 101 can help with the orientation. A first view box 103 can provide information about the total degree of bending of the end section 11, and the portion of the bending which has been achieved either in autonomous mode “Auto” or in the manual mode “Man”. A second view box can inform the user about the actual operation mode. Further information 105, 106 can be provided if the orientation of the tip is 90° or 0°.

In further information boxes 107 and 108 the direction and other information can be provided for the user.

As mentioned above, a miniaturized CCD camera can be located on the anterior surface of the tip 12 or alternatively on a pre-terminal segment 10 to facilitate recognition of the tip position in relation to the anatomical structures. It points towards the tip 12 and displays on the screen 4 an image, preferably a video image, as a front-view of the intubation stylet. An important aspect of this arrangement is that in the lower part of the image, the terminal end of the tip 12 of the intubation stylet is steadily visible. By this configuration, the user may see and visually confirm the passage of the tip through the vocal cords into the trachea. This is the moment to terminate autonomous mode and to switch to manual mode by the user.

The distance of the camera lens of the first camera 14 to the distal edge of the front side 12b of the tip 12 can be optimized in a way that it permits the best possible viewing of the tip 12 when it enters the larynx.

The tip 12 can touch tissue. Therefore, in front surface position the view of the camera could be disturbed. Further to this, there is more space for an LED-Array for light detection when the first camera is positioned in a back-layered position. However it is possible to have a second camera at the front of the tip 12, such as for redundancy and calibration reasons.

A further aspect of the invention is deactivation and overriding the autonomous steering function. A possible option to deactivate the autonomous mode can be provided by tilting the joystick. When this is done, the autonomous mode is deactivated and the manual mode is activated so that the user takes control over the orientation of the tip 12. Alternatively, the user can activate a switch with the alternate positions: automatic and manual.

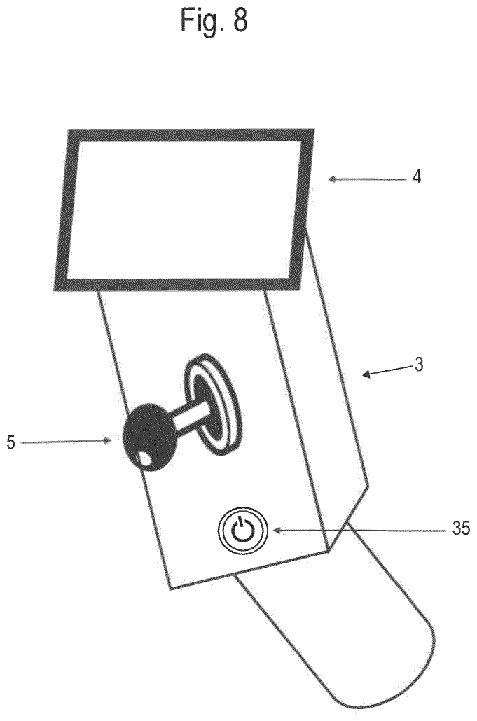

A control unit 3 comprising a screen 4 and a Joystick 5 is shown in FIG. 8. An On/Off button 35 for energizing the intubation stylet or at least the screen 4 is further provided. An optional button (not illustrated) for a retraction movement of the tip 12 can also be provided at the control unit 3.

The joystick 5 with the handle is used to modify the shape of the bending segment according to the user's judgment according to what he sees on the video-screen. This is necessary to avoid further autonomous bending towards the light source after this becomes not necessary anymore or even misleading. From inside the airway, the maximum of light intensity appears anteriorly. This happens as soon as the tip 12 has entered the larynx. From this point on, light guiding is not necessary anymore, and it even might be misleading because inside the airway the light is more intense in anterior direction, while further advancement of the end section 11 should be towards the carina. Overriding may be initiated by releasing the joystick outward and deactivation of overriding and return to autonomous steering may be initiated by applying an axial inward pressure on the joystick, independently into which position it is already tilted.

The joystick 5 can stay in user activated and therefore overriding in the manual mode by default and is kept it in this position by a spring. Full and exclusively autonomous mode is permitted when the joystick is pressed and kept inward by overcoming the opposing spring force.

The stylet is further provided with an on-/off-bottom 6 for switching the stylet and the screen on or off.

Movements of the tip dictated by the user (during overriding) begin at the last autonomously achieved shape. The resulting shape change is calculated by the microprocessor by comparing with a predefined shape of a database and added to the preexisting (last autonomous) shape. The size of additional, user applied shape changing, as expressed in percentage of freedom of movement as compared to the autonomous mode, is a matter of optimization by experiments, and may range from 10 to 100%.

A further aspect of the invention is the in-screen graphic illustration for the tip direction and bending angle.

To provide further information for the actual shape of the flexible segment to the user, an inbuilt graph into the screen view indicates the dimensions of the bending in numeric values and analogue graphical representation. The bending angle can be determined by distance measurement and/or strain measurement. This can be achieved for example by a hose that can be positioned over the segments 7-10 and 12 and their joints, wherein a number of strain gauges are positioned circumferential on the hose at each joint. Other distance measurements such as optical measurement, magnetic measurement or piezo-foil-measurement can be used to determine a bending angle of each individual joint.

A further possibility is to determine the direction in circular grading as a number 001-360° and by a visible line growing from the center into the respective direction. The length of the line indicates the extent (angle) of the bending. As soon as the maximal bending of 90° has been achieved, a red spot in a corner becomes visible. The in-screen projected line is composed by two differently colored segments (e.g., orange for the autonomous component, yellow or white for the manually added component).

As soon as the maximum bending is achieved, a small red flashlight may appear in the corner of the screen. Direction and extent of the bending is graphically represented on the screen in an overlaid head-up display mode both ways, in an analog fashion (by the mentioned lines) and digital fashion by projecting the direction (1-360°) and the bending angle (0-90°) as numerals in the head-up display.

A further aspect of the inventive intubation stylet is an automated airway anatomy recognition including the recognition of vocal cords, tracheal lumen and carina.

As described above, the inbuilt computer or microprocessor of the control unit 3 can be equipped with a software for recognition of the trachea as the right anatomical feature that has been entered and/or by indicating denied recognition if not entered. Typical and distinct morphological features that might contribute to a positive recognition of the airway anatomy including the inner trachea lumen such as are parallel tracheal rings, longitudinal furrows as characteristic feature of the “pars membranacea”, a tunnel like cross sectional shape of the lumen, and the characteristic appearance of the carina.

By applying a comparative method, such as a “fuzzy logic” that calculates the overall probability for having recognized these features would deliver a positive decision for an inside trachea position if a certain threshold value has been passed or denial of tracheal position until the threshold is not passed. In the first case a signal can be delivered to the user and/or an automatic change into the manual mode can be initiated.

In general, the trachea has basically the same form for each patient, including some deviations. It can be described as a tunnel with numerous arcs and two black holes at the end. This unique geometry be used as for an automated recognition of the position and of the distance of the tip 12.

A further aspect of the invention can be the automatic determination of the depth of insertion recognition.

A single photo diode 34 or a plurality of photo diodes can be provided on the anterior surface of the end section 11 at a distance of approximately 20 mm from the tip 12 and oriented perpendicularly to the front surface 12b that senses the light coming from an external light source (or its equivalent). The position can be at the outer surface of a segment 7-10 of the end section 11. While advancing the scope, the light intensity sensed by this photo diode has to increase. After passing beyond the point of highest light intensity, the microprocessor recognizes that the shaft 2 is sufficiently deep in the airway and the tracheal tube might be positioned while the end section 11 may be retracted and removed.

This feature can be added by a sensing of the applied movements e.g., by an external reference point, which can be an inbuilt part of a simultaneously used laryngoscope blade, which is anyway used to open up the intubation pathway, so that the microprocessor also knows when the end section 11 is moved forward, backward or is stationary, as well as the distance in which it has been already forwarded. In combination with the course of the sensed light intensity, it can calculate and recognize the optimal depth of insertion into the trachea.

In a further aspect of the invention a force sensor can be applied to the tip 12. Such a force sensor can detect a resistance, such as the contact with a tissue by comparing the force with a threshold value. The movement of the tip 12 can be registered by the control unit 3. In autonomous mode, a retraction movement can be performed in the same way it has been advanced by applying a predefined force on the tension cables and on the elastic rod 16.

In manual mode the retraction mechanism can be realized in a mechanical way, as described above.

In combination with the aforementioned retraction mechanism, the end section can move backwards for example by 20% in both the autonomous mode and the manual mode.

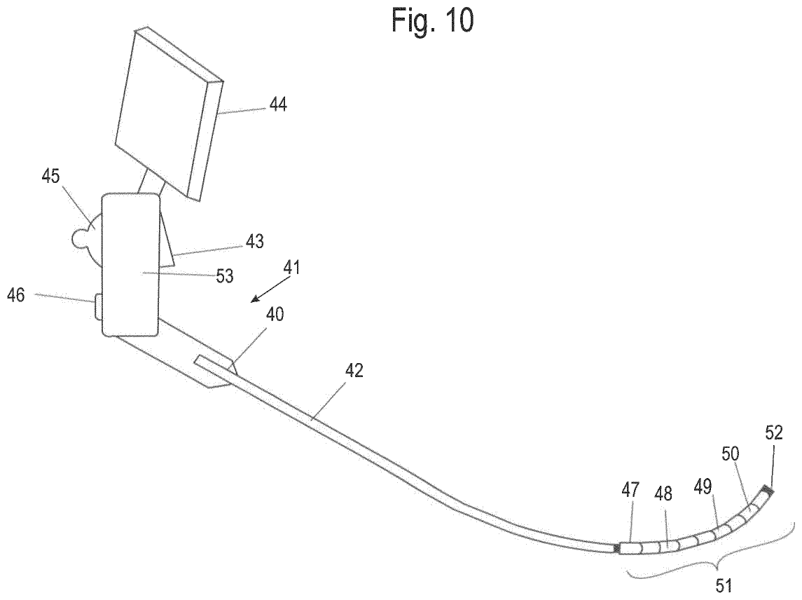

FIG. 10 discloses a second embodiment of an inventive intubation stylet 41 according to the invention. The stylet comprises identically to the stylet of FIG. 1 a shaft 42 and a control unit 43 with a screen 44 for visualization of the position of a tip 52 of the shaft 42 of the intubation stylet 41.

The tip 52 is part of a bendable end section 51 comprising a plurality of linked segments 47-50 and 52. Each segment can be bend to a certain angle to a neighboring segment. The control unit 43 is further provided with an actuating element 45, such as a joystick, for the bending movement of the tip 52 or any other orientation of the tip 52 relative to the shaft 42. The control unit 43 may further comprise an on-/off-Bottom 46 to switch the stylet off or on.

The control may be provided by the actuating element 45 in the same way as described in FIG. 1-9.

The control unit 43 may further comprise a switch 53, such as a monostable switch, which function will be explained.

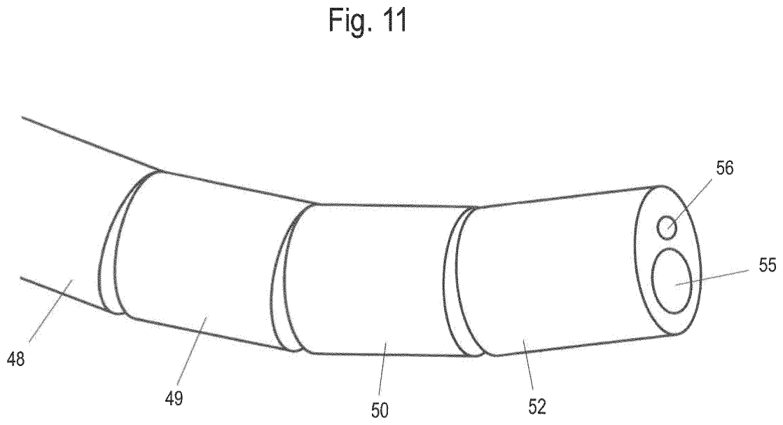

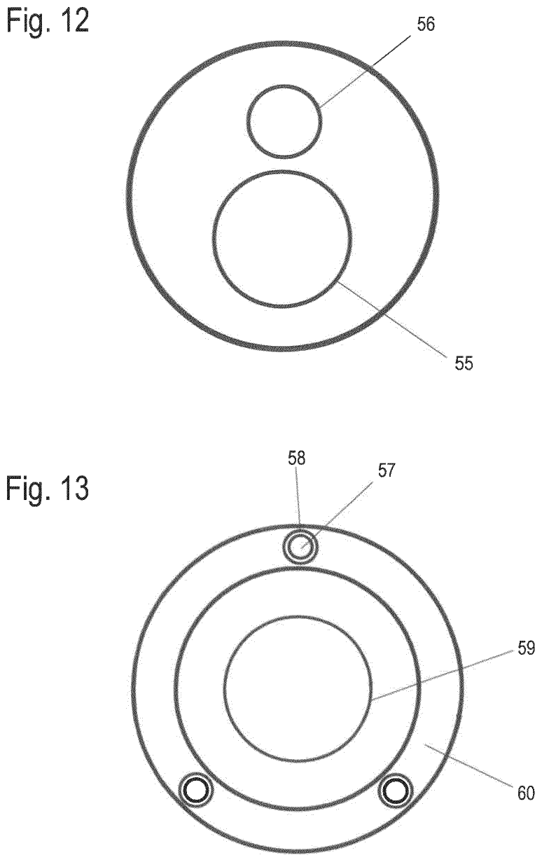

In FIGS. 11 and 12, the tip 52 of the stylet 41 comprises a camera 55, preferably a terminal camera, and a light source 56, especially for visible light, at the end section, especially at the tip 52 of the stylet 41.

The connection between the shaft 42 and the control unit 43 could be an ISO-connection, shaped to hold the tracheal tube. A suitable connection is a 15 mm ISO-connection.

FIG. 13 discloses a cross sectional view in the shaft 42 or any of the segments 47-50 or 52. On an annular area 60 of the shaft or segments is provided with channels 58. Inside the channels 58 suitable tension cables 57 for the control of the bending motion of the end section 51 are provided.

Further to this, FIG. 13 discloses an electrical connection for the camera 55 and the visible light source 56 depicted in FIGS. 11 and 12.

The first and the second embodiment can both be operated in a first and in a second operation mode as described above.

This constitutes different embodiments for the automatic mode (AM) i.e. there are different strategies to use the joystick signal as a user correction input in the automatic mode.

The stylets 1 and 41 are able to operate in two different modes:
Manual mode (MM)
Autonomous mode (AM)

In manual mode, the user uses a 2-DOF (Degree of Freedom) mechanical interface (e.g., a joystick) to generate a velocity command of the tip of the device.

In automatic mode the system automatically computes and generates the velocity command of the tip to steer the stylet tip 12, 52 toward the light source that constitutes the target location.

The joystick can be used to correct the behavior of the device in automatic mode through several strategies that constitutes as many different embodiments of this mode:

Option a) The joystick does not have any effect in automatic mode Option b) The joystick is used to correct the velocity commands automatically computed by the automatic mode (i.e., the two commands are added) Option c) The joystick is used to correct the target location in the camera image Option d) Any action on the joystick over a given threshold automatically overrides the automatic mode and put the device back to manual mode

The switch between the operating modes can be realized as followed. The manual mode is a default mode and automatic mode is to be actively triggered by the user.

To this extend, the automatic mode could be activated by maintaining a monostable system e.g., a monostable switch 53 as depicted in FIG. 10.

Within its unstable state e.g., inward pressure on the monostable switch 53 preferably with the index finger opposed to the thumb used to control the joystick. The manual mode is activated by default when the monostable system is within its stable state e.g., no pressure is applied on the monostable switch 53.

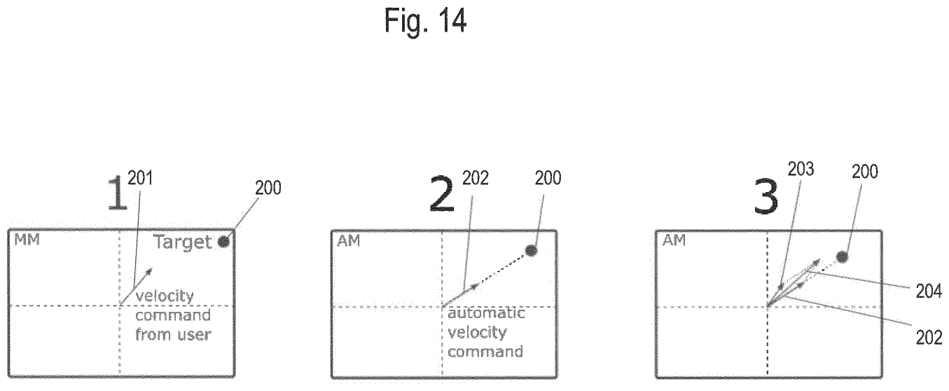

In the following, an example of procedure is proposed for the second embodiment depicted in FIG. 10 for the automatic mode. The procedure is depicted in FIG. 14.

The procedure starts in a first step in manual mode with the monostable switch 53 inactive. The user try to reach the target dot 200 in the screen. The user controls the joystick to generate a velocity command (arrow 201) to move the tip toward the target.

In a second step, the user activates the automatic mode with a maintained pressure on the switch 53 and without any control on the joystick 45. In automatic mode, the control unit computes an automatic velocity command (arrow 202) in the direction of the target 200.

If the user is unsatisfied with the velocity generated by the control unit 43 and decides bring a manual correction with the joystick (arrow 203), that is added to the automatic velocity command. The total velocity command is the addition of both components (arrow 204).

FIG. 14 explains a user-corrected automatic mode in the context of the current invention. As can be derived from the aforementioned text, the first operation mode comprises the automatically guidance of the orientation of the tip by bending towards a direction and the manual correction. So the user corrected mode can be understood as a function, preferably a sum, of forces applied by an drive through the control unit and forces applied by the user, preferably by using the handheld and the actuated pull system.

As can be seen in FIGS. 5, 6 and 11 the longitudinal length of the segments 7-10, 12, 47-50, 52 of the end section including the tip 12, 52 is nearly equal for each of the segments including the tip. This means should not vary more than 20% compared to the neighboring segment of the bendable end section, otherwise a bending motion inside the trachea is largely hindered.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-14. (canceled)

15. An intubation stylet, comprising:
a shaft, comprising a bendable end section with a tip and a camera at the tip for providing an image of a part of an environment of the tip;
a control unit configured to control of a bending motion of the bendable end section, wherein the control unit is configured to operate in at least two operation modes,
wherein, in a first operation mode of the two operation modes, the bending motion of the end section is guided automatically by an orientation of the tip,
wherein, in a second operation mode of the two operation modes, the bending motion of the end section is controlled by manual control of a user without an automatically guidance, and
wherein the control unit is further configured to operate in the second operation mode when the first operation mode is inactive.

16. The intubation stylet of claim 15, further comprising:
an actuated pull system configured to control the bending motion in the second operation mode.

17. The intubation stylet of claim 15, wherein the control unit is configured to automatically switch from the first operation mode to the second operation mode based on image recognition of typical features of an upper airway in an image recorded by the camera at an actual position, wherein the control unit is configured to localize anatomical features in the image using computer vision, wherein the control unit is configured to determine a value representing the actual position or orientation of the tip based on the localization of anatomical features in the image, and wherein the control unit is configured to switch from the first operation mode to the second operation mode if the determined value representing the actual position or orientation of the tip has reached, exceeded, or under-cut a threshold value.

18. The intubation stylet of claim 15, further comprising:
a handheld device configured for the manual control by the user,
wherein the control unit is configured to override the first operation mode by the manual control of the user in such a way that the automatically guiding of the orientation of the tip by bending towards a direction is correctable manually by the user using the handheld device.

19. The intubation stylet of claim 18, wherein the overriding is triggerable by a user decision or by a collision of the end section with an obstacle, which is sensed by sensing a resistance by a force sensor.

20. The intubation stylet of claim 15, wherein the control unit is configured to determine a direction in which the tip is oriented either by detecting a direction of an incoming signal by a sensor element or by an image recognition based on the image recorded by the camera.

21. The intubation stylet of claim 18, wherein the control unit, in the first operation mode, is configured to apply a manual correction to the automatic guidance of the orientation of the tip, wherein the manual correction is a sum of forces applied by an electric motor and forces applied by the user using a handheld device and an actuated pull system.

22. The intubation stylet of claim 15, wherein the bendable end section of the shaft comprises a plurality of joint segments that include the tip, wherein each of the plurality of joint segments has a longitudinal axis, wherein each of the plurality of joint segments is joint tiltable to a neighboring one of the plurality of joint segments such that a longitudinal axis of the one of the plurality of joint segments and the neighboring one of the plurality of joint segments define a bending angle, and wherein the bending angle between two neighboring ones of the plurality of joint segments increases towards the tip of the intubation stylet.

23. The intubation stylet of claim 15, wherein a length of the plurality of joint segments including the tip does not vary more than 20% compared to a neighboring one of the plurality of joint segments of the bendable end section.

24. An intubation system comprising:
a intubation stylet, which comprises
a shaft, comprising a bendable end section with a tip and a camera at the tip for providing an image of a part of an environment of the tip;
a control unit configured to control of a bending motion of the bendable end section, wherein the control unit is configured to operate in at least two operation modes,
wherein, in a first operation mode of the two operation modes, the bending motion of the end section is guided automatically by an orientation of the tip,
wherein, in a second operation mode of the two operation modes, the bending motion of the end section is controlled by manual control of a user without an automatically guidance,
wherein the control unit is further configured to operate in the second operation mode when the first operation mode is inactive,
wherein the intubation stylet is a first module of the intubation system for the insertion through the mouth of a patient,
wherein the tip comprises a sensor element configured to detect a direction of an incoming signal, and
wherein in the first operation mode the bending motion of the end section is guided automatically by orienting the tip towards the direction of the signal based on at least one control-signal generated by the sensor element;
a light source, which is a second module of the intubation system, configured for external adaptation at the throat of a patient for emitting the incoming signal.

25. The intubation system of claim 24, wherein the light signal source emits a signal that corresponds to the incoming signal, wherein the signal is a near-infrared signal.

26. A method to operate the intubation stylet, which comprises a shaft, comprising a bendable end section with a tip and a camera at the tip for providing an image of a part of an environment of the tip; a control unit configured to control of a bending motion of the bendable end section, wherein the control unit is configured to operate in at least two operation modes, the method comprising:
controlling, in a first operation mode of the two operation modes, the bending motion of the end section by an autonomous orientation of the end section; and
controlling, in a second operation mode of the two operation modes, the bending motion of the end section is controlled by manual control of a user,
wherein the second operation mode is performed only when the first operation mode is inactive, and
wherein the first operation mode is switched to the second operation mode based an image recognition, which comprises
recording, by the camera, an image of a part of an environment of the tip at an actual position;
localizing, by the control unit using computer vision, anatomical features inside a trachea;
determining a value representing the actual position or orientation of the tip based on the localizing of the anatomical features; and
switching from the first operation mode to the second operation mode if the determined value has reached, exceeded, or under-cut a threshold value.

27. The method of claim 26, wherein the bending motion of the end section is continuously measured and displayed at a screen.

28. The method of claim 26, wherein the bending motion of the end section is controlled either in the first or second operation mode, wherein advancement of the intubation stylet in the trachea is only performed manually.
